(19) United States
(12) Patent Application Publication
(10) Pub. No.: US 2023/0382728 A1
(43) Pub. Date: Nov. 30, 2023

(54) INTEGRATED CARBON CAPTURE AND OLEFINS PRODUCTION PROCESS

(71) Applicant: Kellogg Brown & Root LLC, Houston, TX (US)

(72) Inventor: Michael J. Tallman, Houston, TX (US)

(73) Assignee: Kellogg Brown & Root LLC, Houston, TX (US)

(21) Appl. No.: 18/324,034

(22) Filed: May 25, 2023

Related U.S. Application Data
(60) Provisional application No. 63/345,596, filed on May 25, 2022.

(51) Int. Cl. C01B 3/34; C07C 1/12

(52) U.S. Cl. CPC C01B 3/34; C07C 1/12; C01B 2203/0805; C01B 2203/061; C01B 2203/1205; C01B 2203/86

(57) ABSTRACT

Carbon capture of carbon dioxide (CO₂) gives an extracted CO₂ stream that is reacted with hydrogen to produce methanol (MeOH), which can in turn be fed to catalytic production of olefins such as ethylene and propylene to give an integrated process.

### CROSS REFERENCE TO RELATED APPLICATIONS

This application claims the benefit of U.S. Provisional Application No. 63/345,596, filed May 25, 2022, the contents of which are incorporated herein by reference in their entirety.

### TECHNICAL FIELD

The present invention relates to systems and methods for capturing carbon from flue gas. More particularly, to systems and methods for capturing carbon from flue gas and then reacting it with hydrogen to form methanol which can then be converted to olefins.

### BACKGROUND

There is great global interest in carbon emission reduction methods. Approaches include carbon capture and storage (CCS) or carbon capture and sequestration, which are processes of capturing carbon dioxide (CO₂) before it enters the atmosphere, transporting it, and storing it for centuries or millennia. Typically, the CO₂ is captured from large point sources, such as coal-fired power plants, chemical plants, or biomass power plants, and then stored in an underground geological formation. A goal is to prevent the release of CO₂ from heavy industry with the intent of mitigating potential climate change effects.

CO₂ can be captured directly from an industrial source, such as a cement kiln, using a variety of technologies including, but not necessarily limited to, absorption, adsorption, chemical looping, membrane gas separation, and/or gas hydration. As of about 2020, about one thousandth of global CO₂ emissions are captured. Most projects are industrial.

Storage of CO₂ is envisaged either in deep geological formations, or in the form of mineral carbonates. Geological formations are currently considered the most promising sequestration sites. It has been estimated that North America has enough storage capacity for more than 900 years' worth of CO₂ at current production rates. A general concern is that long-term predictions about submarine or underground storage security are very difficult and uncertain, and there is still risk that some of the stored CO₂ will leak into the atmosphere.

It would be desirable to find a productive way of utilizing the extracted CO₂ instead of simply storing it, which may be described as a carbon capture and utilization process.

### SUMMARY

An integrated system for carbon capture and olefins production, where the integrated system may include a carbon capture unit configured to extract a CO₂ stream from a flue gas source feed; a CO₂ reactor downstream of the carbon capture configured to react CO₂ from the CO₂ stream with hydrogen to produce a methanol product stream; and a catalytic olefin reactor downstream of the CO₂ reactor configured to receive the methanol product stream and an additional hydrocarbon feed and catalytically react methanol from the methanol product stream to give an intermediate olefin stream.

In examples, the additional hydrocarbon feed is mixed with the methanol product stream before it enters the catalytic olefin reactor.

The additional hydrocarbon feed may include C4-C12 paraffinic, olefinic fluid hydrocarbon feed stock streams, or any combination thereof.

In examples, the system may include at least one pyrolysis furnace adapted to feed at least one olefin to the recovery unit.

In examples, the catalytic olefin reactor may include integrated gas oil and light olefin cracking zones having a petrochemical product stream comprising ethylene and/or propylene.

In examples, the catalytic olefin reactor may include a fluid catalytic cracker comprising a first riser reactor to maximize gasoline range molecules recycled to a second riser reactor to maximize ethylene and propylene yields.

In examples, the system may include a recovery unit for separating the intermediate olefin stream into at least one olefin product stream.

A process for integrated carbon capture and olefins production that may include supplying flue gas to a carbon capture unit; extracting CO₂ from the flue gas in the carbon capture unit to generate a CO₂ stream; reacting the CO₂ from the CO₂ stream with hydrogen to produce a methanol product stream; reacting methanol from the methanol product stream in the presence of a catalyst and additional hydrocarbons to produce an intermediate olefin stream.

In examples, the process may include introducing an additional hydrocarbon feed to the methanol product stream prior to reacting methanol in the presence of a catalyst to produce the intermediate olefin stream.

In examples, the additional hydrocarbons may include C4-C12 paraffinic, olefinic fluid hydrocarbon feed stock streams, or any combination thereof.

In examples, least one olefin from the olefin stream may be fed to a recovery unit.

In examples, reacting methanol from the methanol product stream may include cracking the methanol thereby producing petrochemicals comprising ethylene and/or propylene.

In examples, reacting methanol from the methanol product stream may include producing gasoline range molecules in a first riser reactor and recycling them to a second riser reactor to yield ethylene and propylene.

In examples, the intermediate olefin stream may be fed to a recovery unit and fractionating the intermediate olefin stream into at least one olefin product stream.

### BRIEF DESCRIPTION OF THE DRAWING

FIG. 1 is a non-limiting, schematic block flow diagram of an integrated system for carbon capture and olefins production as described herein.

It will be appreciated that the drawing is a schematic illustration and that the invention is not limited to the design, proportions, or specific equipment shown in the drawing.

### DETAILED DESCRIPTION

In examples, described herein is an integrated system for carbon capture and olefins production. It has been discovered that CO₂ extraction technology can be combined with process technology to convert CO₂ to methanol (MeOH or CH₃OH), and a catalytic olefins process to covert the methanol to propylene and ethylene. In examples, the catalytic olefins process may not specifically be designed to convert methanol directly to olefins.

In examples, an integrated process as described may be a combination of:
1) Extracting CO₂ from flue gas,
2) Converting the extracted CO₂ to methanol, and
3) Feeding the methanol to a catalytic olefins converter for production of olefins such as propylene and ethylene.

In examples, by using such an integrated system, CO₂ emissions can be reduced or eliminated while using the CO₂ for producing olefins.

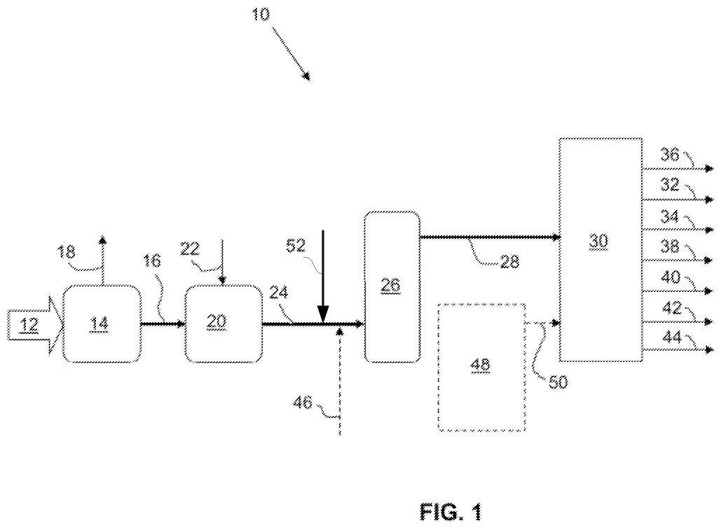

FIG. 1 illustrates a non-limiting, schematic block flow diagram of an example of an integrated system 10 for carbon capture and olefins production as described herein.

In examples, a flue gas source provides flue gas feed 12 to a carbon capture unit 14. As used herein, a flue gas can refer to a byproduct from any process that requires fuel to provide heat, including, but not limited to, coal-fired power plants, chemical plants, or biomass power plants, steam cracking plants, catalytic olefin processes, propane dehydrogenation plants, etc.

In examples, the carbon capture unit 14 can use any technology for extracting CO₂ from a flue gas. For example, the carbon capture unit 14 may employ any one or more of absorption, adsorption, chemical looping, membrane gas separation, gas hydration, and/or extraction with an amine or hot potassium carbonate. Other technologies for CO₂ extraction may also be used. Carbon extraction methods to recover CO₂ from flue gas are generally known. In examples, a CO₂ extraction process can employ a solvent to remove the CO₂ from the flue gas. In examples, the solvent can be an amine or hot potassium carbonate. Other solvents may also be used.

In examples, the carbon capture unit 14 can extract a CO₂ stream 16 and emit a residual flue gas 18 via a stack. In examples, the residual flue gas 18 may be released to the atmosphere if it is sufficiently free of CO₂. In the alternative, the residual flue gas 18 may be collected, stored, and/or undergo sequestration or other processing.

In examples, the CO₂ stream 16 may be directed to a CO₂ reactor 20. In examples, the CO₂ stream 16 may optionally be combined with a hydrogen stream 22 prior to entering the CO₂ reactor 20. In examples, the CO₂ reactor 20 may be configured to react the CO₂ with hydrogen to produce a methanol product stream 24.

In examples, the methanol product stream 24 from CO₂ reactor 20 may be directed to a catalytic olefin reactor 26. In examples, the catalytic olefin reactor 26 may be adapted to catalytically react the methanol to give an intermediate olefin stream 28.

In examples, the catalytic olefin reactor 26 may not specifically be designed to convert methanol directly to olefins. In examples, the catalytic olefin reactor 26 may include a fluidized bed reactor. In examples, the catalytic olefin reactor 26 may be configured to receive one or more feed streams. In examples, the catalytic olefin reactor 26 may be configured to produce olefins such as ethylene and propylene. In examples, additional products may also result from catalytic olefin reactor 26. In examples, it may be possible to improve the overall production of valuable products, for example aromatics or components with C4 value chain, in catalytic olefin reactor 26 by feeding the methanol product stream 24 to the catalytic olefin reactor 26.

In examples, the methanol product stream 24 is mixed with an additional feed either before entering the catalytic olefin reactor 26 and/or inside the catalytic olefin reactor 26. In examples, the additional feed is a hydrocarbon feed. In examples, the additional feed may be introduced via additional feed line 52. In examples, the hydrocarbon feed introduced via additional hydrocarbon feed line 52 may include a stream of one or more hydrocarbons. In examples, the one or more hydrocarbons may include C4-C12 paraffinic and olefinic fluid hydrocarbon feed stock streams. In examples, the one or more hydrocarbons may include straight-run naphtha, cracked naphtha (such as from an FCC or coker unit), steam cracker byproduct C4, C5, C6-C8 non-aromatics, C4 LPG, any combinations thereof as well as other hydrocarbon streams. In examples, as illustrated, the hydrocarbon feed may be mixed with the methanol product stream 24 before entering the catalytic olefin reactor 26. In examples, the methanol product stream 24 is included as a portion of the feed to the catalytic olefin reactor 26. In examples, the methanol product stream 24 may provide up to 80% of the total feed to catalytic olefin reactor 26.

In examples, a secondary feed line 46 may further be used to introduce secondary feed material into the methanol product stream 24. Suitable secondary feed materials include, but are not necessarily limited to, methanol from other sources, other oxygenates such as ethanol, butanol, various other alcohols, aldehydes and ketones, certain hydrocarbon streams derived from plastics recycling conversion processes, or certain other renewable bio-feeds.

In examples, the process conditions of catalytic olefin reactor 26 may be set to achieve conversion of the methanol from methanol product stream 24 along with any added feed. In examples, the catalytic olefin reactor 26 may operate at a temperature ranging from about 500° C. to about 700° C. In examples, the catalytic olefin reactor 26 may operate at a pressure of up to about 10 bars. In examples, the catalytic olefin reactor 26 may include a catalyst to oil or to olefin ratio of up to 1:50.

In examples, the catalytic olefin reactor 26 may be configured to produce low molecular weight hydrocarbons, including propylene, and ethylene, from C4-C12 paraffinic and olefinic fluid hydrocarbon feed stock streams, which may optionally contain one or more aromatic constituents. In examples, the feedstock streams to catalytic olefin reactor 26 under hydrocarbon conversion conditions may be contacted with an activated catalyst at a residence time of less than five (5) minutes in a dilute phase transfer line fluidized bed reactor. In examples, the system may optionally include a separation process to purify the propylene and ethylene products.

In examples, the catalytic olefin reactor 26 may be integrated with one or more additional components or units.

In examples, the process of catalytic olefin reactor 26 may include the integration of a catalytic olefin cracker as described above with a pyrolytic cracking zone. In examples, the pyrolytic cracking zone may enable cracking of light alkanes. In examples, the light alkane may include, but be not limited to, ethane, propane and/or butane. In examples, the light alkane may be derived from the catalytic cracking process, and/or from a fresh source. In examples, the process may optionally include the separation process to purify propylene and ethylene products.

In examples, the process of catalytic olefin reactor 26 may include the integration of gas oil and light olefin catalytic cracking zones, optionally with a pyrolytic cracking zone to maximize efficient production of petrochemicals as for example generally described in U.S. Pat. No. 7,128,827, which is incorporated herein by reference in its entirety. In examples, integration of the units in parallel may lead to the production of an overall product stream with enhanced amounts of ethylene and/or propylene by routing various feedstreams and recycle streams to the appropriate cracking zone(s), e.g., ethane/propane to the steam pyrolysis zone and C4-C6 olefins to the light olefin cracking zone. In examples, such integration may enhance the value of the material balances produced by the integrated units.

In examples, the catalytic olefin reactor 26 may include a natural transition from the traditional fuel mode of fluid catalytic cracking (FCC) operations where gasoline may be maximized to high production of light olefins. In examples, using proven FCC technologies, by adding a second reactor riser mixed C4s and/or light naphtha, along with other extraneous low valued streams, may be recycled to the second riser where additional cracking may occur. In examples, this may create propylene and/or butylene from heavier compounds. In examples, ethylene may be also produced. In examples, the ratio of C2/C3 olefins can be increased by increasing the severity of the second riser.

In examples, a way to maximizing olefins from an FCC unit may be to maximize gasoline range molecules. In examples, the process may use the principle of “decoupled reactions” to maximize olefins from the FCC unit. For example, the process may use a two-riser design whereby the amount of gasoline range molecules is maximized in the primary riser and the stream is selectively recycled back to the second riser to further concentrate the propylene and ethylene yields. This process may operate in the conventional operational regime of a conventional FCC unit in terms of operating temperatures and hydrocarbon partial pressure which can advantageously result in smaller equipment size.

In examples, the process may be designed to maximize the production of propylene from traditional FCC feedstocks and selected naphthas. For example, the process can increase propylene yield relative to that produced by conventional FCC units by combining the effects of catalysts and hardware, including a second high severity riser designed to crack surplus naphtha into light olefins.

In examples, during other modes of operation, such as maximum gasoline or maximum distillate, the second high severity riser can be either shut down or used to process additional fresh feed at conditions same as the first riser. Thus, the technology may allow a refiner flexibility for maximizing the production of light olefins without sacrificing seasonal changes in operating modes.

In examples, the intermediate olefin stream 28 from catalytic olefin reactor 26 may be optionally fed to a recovery unit 30 for separating the intermediate olefin stream 28 into at least one olefin product stream.

In examples, the at least one olefin product stream from the optional recovery unit 30 may include, but is not necessarily limited to, an ethylene stream 32 and/or a propylene stream 34. Other recovered streams may optionally include, but are not necessarily limited to, any one or more of hydrogen stream 36, tail gas stream 38, raw C4 stream 40, gasoline stream 42 including benzene-toluene-xylene (BTX), and fuel oil stream 44, or any combination thereof.

In examples, hydrogen stream 36 from recovery unit 30 may be used to provide hydrogen for all or a portion of hydrogen stream 22 for the conversion of the CO₂ stream 16 to methanol.

In examples, tail gas stream 38 from recovery unit 30 may be converted to methanol, which could optionally be one of the streams comprising fuel oil stream 44.

In examples, the system may include a pyrolysis furnace 48 for cracking light recycle streams including, but not necessarily limited to, ethane and/or propane, to supply additional fresh olefin feed 50 to the recovery unit 30 to contribute to the production of the various product streams. Fresh olefin feed 50 may include, but is not necessarily limited to, ethylene and/or propylene.

It will be appreciated that the system and method described herein can simultaneously reduce or eliminate CO₂ from a process while also using the CO₂ to produce useful olefins.

In the foregoing specification, the invention has been described with reference to specific embodiments thereof. However, the specification is to be regarded in an illustrative rather than a restrictive sense. For example, equipment, risers, reactors, CO₂ sources, carbon capture technology, columns, process streams, processes, reactants, saturated hydrocarbons, catalytic processes for olefin production, olefins and other products, catalysts, and operating conditions falling within the claimed or disclosed parameters, but not specifically identified or tried in a particular example, are expected to be within the scope of this invention.

The present invention may be practiced in the absence of an element not disclosed. In addition, the present invention may suitably comprise, consist or consist essentially of the elements disclosed. For instance, there may be provided an integrated system for carbon capture and olefins production, where the integrated system comprises, consists essentially of, or consists of a flue gas source feed supplying a carbon capture unit, where the carbon capture unit extracts a CO₂ stream and emits a flue residue gas, a CO₂ reactor receiving the CO₂ stream and a hydrogen stream, where the CO₂ reactor is adapted to react the CO₂ and hydrogen to produce a methanol product stream, and a catalytic olefin reactor receiving the methanol product stream adapted to catalytically react the methanol to give an intermediate olefin stream. The integrated system optionally comprises, consists essentially of, or consists of recovery unit for separating the intermediate olefin stream into at least one olefin product stream.

There may be further provided a process for integrated carbon capture and olefins production, the process comprising, consisting essentially of, or consisting of supplying flue gas to a carbon capture unit, extracting CO₂ from the flue gas in the carbon capture unit to give a CO₂ stream and a flue residue gas, feeding the CO₂ stream and hydrogen to a CO₂ reactor and reacting the CO₂ and hydrogen to give a methanol product stream, feeding the methanol product stream to a catalytic olefin reactor, reacting methanol from the methanol product stream in the presence of a catalyst to produce an intermediate olefin stream. The process optionally comprises, consists essentially of, or consists of feeding the intermediate olefin stream to a recovery unit and fractionating the intermediate olefin stream into at least one olefin product stream.

The words “comprising” and “comprises” as used throughout the claims, are to be interpreted to mean “including but not limited to” and “includes but not limited to”, respectively.

As used herein, the word “substantially” shall mean “being largely but not wholly that which is specified.”

As used herein, the singular forms “a,” “an,” and “the” are intended to include the plural forms as well, unless the context clearly indicates otherwise.

As used herein, the term “about” in reference to a given parameter is inclusive of the stated value and has the meaning dictated by the context (e.g., it includes the degree of error associated with measurement of the given parameter).

As used herein, the term “and/or” includes any and all combinations of one or more of the associated listed items.

1. An integrated system for carbon capture and olefins production, where the integrated system comprises:
a carbon capture unit configured to extract a CO₂ stream from a flue gas source feed;
a CO₂ reactor downstream of the carbon capture configured to react CO₂ from the CO₂ stream with hydrogen to produce a methanol product stream; and
a catalytic olefin reactor downstream of the CO₂ reactor configured to receive the methanol product stream and a hydrocarbon feed and catalytically react methanol from the methanol product stream to give an intermediate olefin stream.

2. The integrated system of claim 1, wherein the hydrocarbon feed is mixed with the methanol product stream before it enters the catalytic olefin reactor.

3. The integrated system of claim 2, wherein the hydrocarbon feed comprises C4-C12 paraffinic, olefinic fluid hydrocarbon feed stock streams, or any combination thereof.

4. The integrated system of claim 1, wherein the catalytic olefin reactor comprises integrated gas oil and light olefin cracking zones having a petrochemical product stream comprising ethylene and/or propylene.

5. The integrated system of claim 1, wherein the catalytic olefin reactor comprises a fluid catalytic cracker comprising a first riser reactor to maximize gasoline range molecules recycled to a second riser reactor to maximize ethylene and propylene yields.

6. The integrated system of claim 1, further comprising a recovery unit for separating an intermediate olefin stream from the catalytic olefin reactor into at least one olefin product stream.

7. The integrated system of claim 6, further comprising at least one pyrolysis furnace adapted to feed at least one olefin to the recovery unit.

8. A process for integrated carbon capture and olefins production, the process comprising:
supplying flue gas to a carbon capture unit;
extracting CO₂ from the flue gas in the carbon capture unit to generate a CO₂ stream;
reacting the CO₂ from the CO₂ stream with hydrogen to produce a methanol product stream;
reacting methanol from the methanol product stream in the presence of a catalyst and one or more hydrocarbons to produce an intermediate olefin stream.

9. The process of claim 8, further comprising introducing a hydrocarbon feed comprising the one or more hydrocarbons to the methanol product stream prior to reacting methanol in the presence of a catalyst to produce the intermediate olefin stream.

10. The process of claim 8, wherein the one or more hydrocarbons comprise C4-C12 paraffinic, olefinic fluid hydrocarbon feed stock streams, or any combination thereof.

11. The process of claim 8, wherein the reacting methanol from the methanol product stream further comprises cracking the methanol thereby producing petrochemicals comprising ethylene and/or propylene.

12. The process of claim 8, wherein the reacting methanol from the methanol product stream further comprises producing gasoline range molecules in a first riser reactor and recycling them to a second riser reactor to yield ethylene and propylene.

13. The process of claim 8, further comprising feeding the intermediate olefin stream to a recovery unit and fractionating the intermediate olefin stream into at least one olefin product stream.

14. The process of claim 8, further comprising feeding at least one olefin from the olefin stream to a recovery unit.
